(19) United States
(12) Patent Application Publication
(10) Pub. No.: US 2006/0258003 A1
(43) Pub. Date: Nov. 16, 2006

(54) Culture medium composition, culture method, and myoblasts obtained, and their uses

(75) Inventor: Christian Pinset, Paris (FR)

Correspondence Address: STATTLER, JOHANSEN, AND ADELI LLP, 1875 CENTURY PARK EAST SUITE 1360, LOS ANGELES, CA, 90067, US

(73) Assignee: CELOGOS, Paris (FR)

(21) Appl. No.: 10/538,655

(22) PCT Filed: Dec. 12, 2003
(86) PCT No.: PCT/FR03/03691

§ 371 Date: Dec. 23, 2005

(30) Foreign Application Priority Data
Dec. 13, 2002 (FR) 02/15827

(51) Int. Cl. C12N 5/08

(52) U.S. Cl. 435/366; 435/404

(57) ABSTRACT

The invention concerns a composition of culture medium of progenitor/stem cells derived from muscular tissues containing serum and/or serum fraction of human origin and/or of animal origin of insulin or a derivative thereof, and one or several compound(s) selected among the class of antioxidants and/or of vitamins. The invention also concerns a method for culturing progenitor/stem cells, a method for producing myoblasts capable of being used as cellular/genetic therapy product. The invention aims at optimizing the production of myoblasts from progenitor/stem cells.

The present invention relates to a culture medium composition for progenitor/stem cells originating from muscle tissues, to a culture process for progenitor/stem cells, and a process for producing myoblasts capable of being used as cell/gene therapy products.

Numerous studies relating to cell culture processes have been carried out for the purpose of obtaining cell fractions rich in myoblasts for administration to patients suffering generally from muscular degeneration such as Duchenne muscular dystrophy for example. Many of these have related to the choice of the initial cells, culture conditions, or the cell identification step for example.

In this respect, there can be mentioned the document U.S. Pat. No. 5,538,722 which proposes a process for in vivo synthesis of a muscle protein which results from the integration of a DNA into the myoblasts in culture, these myoblasts having undergone at least 5 cell duplications.

The document U.S. Pat. No. 5,130,141 discloses a process for obtaining myogenic cells originating from culture and also myogenic cells which have been previously cloned, the latter having advantages over the former due to their superior development potential.

The document US-A-2001 0034061 discloses a process for the culture of progenitor cells by controlled use of hypoxic culture in order to promote a specific differentiation.

Finally, the document WO-A-01 94555 proposes providing well-characterized cell populations of muscle origin, suitable and especially prepared for their desired use in cell therapy.

However, there are a more limited number of works which have been carried out on the composition of the culture medium itself with a view to producing a cell population suitable for use in cell/gene therapy.

Among the latter, there is the European Application EP-A-1048724 which relates to a process for the culture of immortalized muscle cell lines, i.e. which have been obtained after a high number of passages, which are used in gene therapy, either in “repairing” defective muscle tissues or as vectors into which one or more genes can be introduced in order to provide a determined product.

The document WO-A-97 00774 teaches a means for improving the taking of a graft by “preconditioning” the myoblasts of the donor in the presence both of a growth factor such as bFGF and an inducer of metalloprotease production, in order to increase the migration distance of the transplanted myoblasts and in order to increase the number of fused myoblasts expressing functional proteins of the muscle.

The document WO-A-99 56785 discloses a process for producing muscle cells which are genetically modified before being injected into the muscle dysfunction sites: this process being intended in particular for treating urinary incontinence.

The document WO-A-01 78754 refers to progenitor cells having long-term in situ survival, which have a particular profile of expression of cell markers and can be used in the treatment of urinary incontinence.

Finally, the document WO-A-02 067867 relates to a process for preparing stem cells by using a cell matrix to bind the latter: it is in particular intended for urinary treatment.

The literature, including all these documents of the prior art mentioned above, thus share the characteristic that the non-supplemented animal serum (non-human, for example bovine or equine) is used during the actual cell culture, the latter probably being considered as a sufficient supply of all the elements necessary for cell proliferation.

Transplantation requires the production of a high number of myoblasts, it is then important to improve this production by starting with progenitor/stem cells originating from muscle tissues. The present invention proposes supplementing the serum (or serum fraction) which is used in the culture medium, thus making it possible to optimize the culture medium. Therefore, it is possible to shorten the culture time. Thus this optimization then makes it possible to use less serum (or serum fraction), which is necessary in the case of human serum, the availability of which is limited and at the same time makes it possible to do without the use of animal proteins, potential sources of contamination by the prion or viruses.

In order to resolve the problem of optimization of the production of myoblasts starting with progenitor/stem cells, the present invention proposes a cell culture medium composition containing:
(i) serum and/or serum fraction of human origin and/or of animal origin
(ii) insulin or a derivative of the latter
(iii) one or more compound(s) chosen from the class of antioxidants and/or vitamins.

Serum and/or serum fraction of bovine origin, preferably of human origin can be used. Advantageously, the concentration of human serum is less than 5% by volume, and still more advantageously between 1% and 3% by volume.

Typically, the insulin derivative is chosen from the class of the IGFs, and vanadate-type insulomimetics.

Advantageously, the vitamin is ascorbic acid, and the antioxidant is N-acetyl-cysteine or selenium.

In an embodiment, one or more compound(s) chosen from the class of FGF-type growth factors can moreover be used. Typically, this growth factor is chosen from the class of the bFGFs, FGF-2 to FGF-10.

In another embodiment, the culture medium can if appropriate comprise a glucocorticoid.

In another embodiment, the culture medium composition moreover comprises lipophosphatidic acid and/or one or more compound(s) from the classes of the EGFs, heregulins, thrombin, PDGF, thyroid hormones and LIF.

The present invention also relates to a process for the culture of progenitor and/or stem cells, in which the composition previously presented is used as culture medium during the cell amplification step.

According to a embodiment, the cell differentiation step is carried out before, during or after the cell amplification step. Typically, the human serum used is autologous with the progenitor/stem cells.

The invention also relates to a process for producing myoblasts by implementation of the process previously presented.

According to an embodiment, the progenitor and/or stem cells are obtained by a step of cell extraction from muscle tissues. Advantageously, the extraction step is carried out by enzymatic digestion.

According to another embodiment harvesting and separation of the cells obtained is carried out. Typically, the harvesting and separation of the cells is carried out by enzymatic digestion followed by centrifugation and/or filtration. The step of enzymatic digestion can moreover be omitted.

According to another embodiment, the suitability of the myoblasts for forming colonies is tested.

According to another embodiment, a cell characterization is carried out. Advantageously, cell cycle markers are used.

According to another embodiment, a step of freezing of the myoblasts is carried out.

The invention also relates to a cell population containing progenitor/stem cells or myoblasts or a mixture of the latter in the culture medium.

According to the invention, the myoblasts produced according to the process previously mentioned can be used for cell therapy purposes. Preferably, they are intended for the preparation of a product intended for the treatment of urinary incontinence or the functional treatment of the small muscles (a non-exhaustive list of these muscles characterized by their small size comprises the sphincters such as the urethral or anal sphincters, the eyelid muscles, the muscles of the fingers and the muscles of the larynx).

According to another embodiment, the myoblasts thus produced are intended for gene therapy.

Finally, the present invention relates to the use of the myoblasts produced in toxicological and/or pharmacological screening. Typically, this screening is aimed at detecting one or more substance(s) involved in rhabdomyolysis.

Other characteristics and advantages of the invention will become apparent on reading the description which follows of the embodiments of the invention, given as examples only and with reference to the drawings which show:

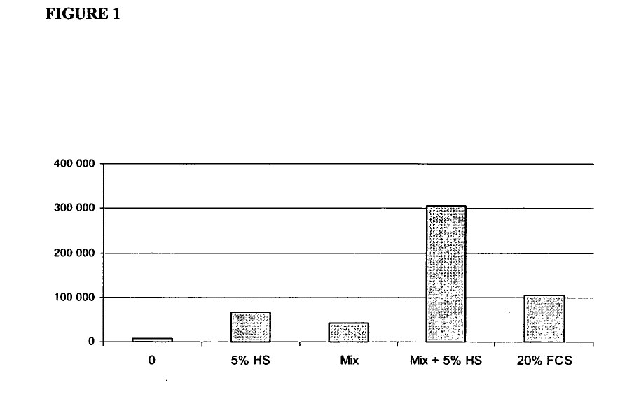

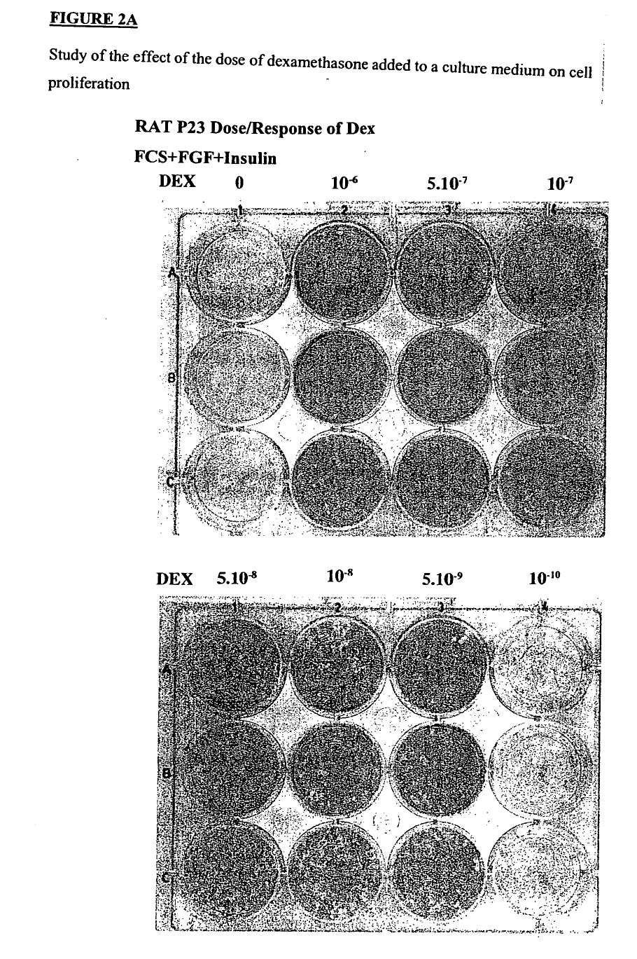

FIG. 1: Histograms representing the number of nuclei of human muscle cells per unit of surface area using culture media: (A) devoid of growth factors; (B) containing 5% human serum according to the invention; (C) FGF+insulin+PDGF+EGF+dexamethasone+thrombin (Mixture M); (D) corresponding to (B)+(C), and “FCS” medium containing 20% foetal calf serum.

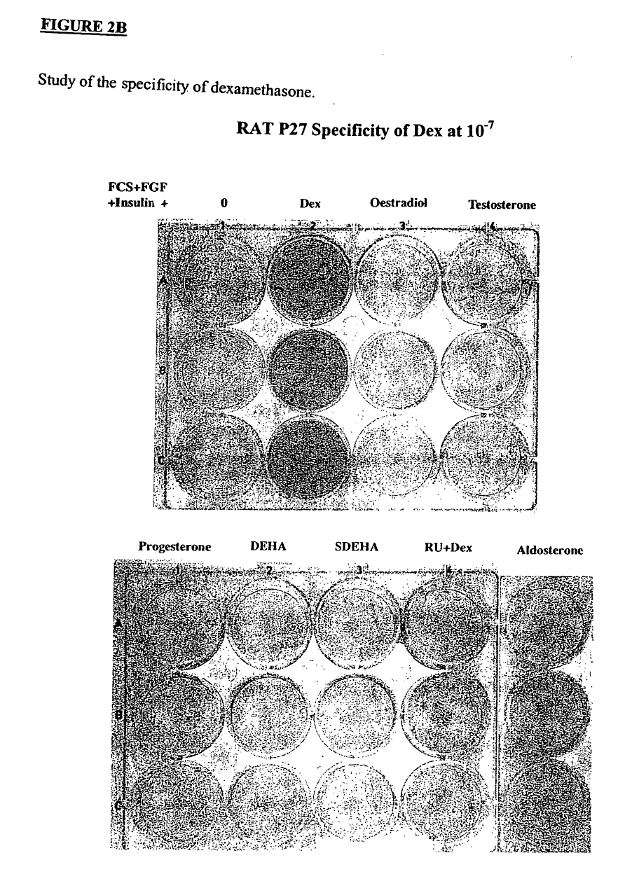

FIG. 2A: Study of the effect of the dose of dexamethasone (concentrations of 0 to 10⁻⁶ M) added to a culture medium containing foetal calf serum (FCS) supplemented by insulin and FGF on the proliferation of rat cells originating from passage 23.

FIG. 2B: Study of the specificity of dexamethasone. In a culture medium containing foetal calf serum (FCS) supplemented by insulin and FGF, the effect of the addition of 10⁻⁷ M dexamethasone, or of another steroid hormone (oestradiol, testosterone, progesterone, DEHA, SDEAH, aldosterone) at a fixed dose (10⁻⁷ M) on cell growth is observed. Dexamethasone is also tested in combination with the antiprogestagen RU486.

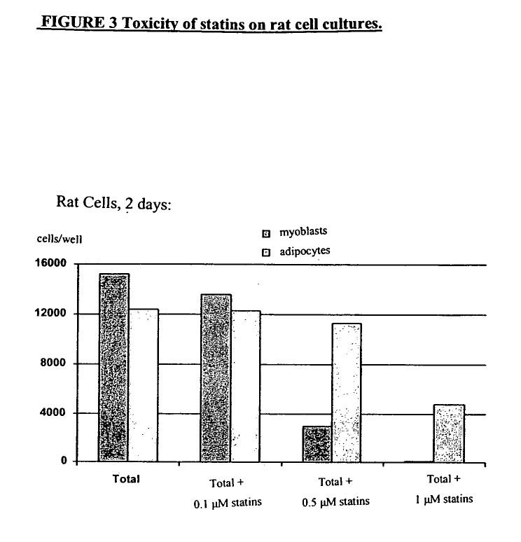

FIG. 3: Comparative study of the toxicity of lovastatin on muscle cells and adipocyte cells.

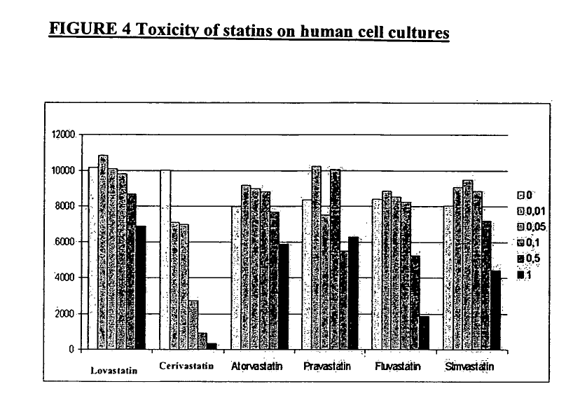

FIG. 4: Comparative study of the toxicity on human muscle cells of the different statins in human clinical use.

In FIGS. 2A and 2B, the intensity of staining (represented here by the dark stains) increases with the cell density. A series of three culture wells was carried out for each concentration.

The present invention relates firstly to a culture medium composition intended for cell proliferation and/or differentiation. This culture medium can in particular be used in order to ensure the proliferation and differentiation of muscle progenitor and/or stem cells in myoblasts. In addition to the basic nutrient medium, this culture medium composition of the invention comprises as a minimum serum of human origin and/or of animal origin, insulin (or one of its derivatives) and an antioxidant and/or a vitamin.

The basic nutrient medium used is buffered with buffers dependent on or independent of the CO₂ concentration. It is preferable to exclude Hepes from the culture medium as buffer as a concentration of 15 mM inhibits the growth of human muscle cells in the long term. The media used are, in the majority of cases, constituted by a mixture of DME-type, Ham's F12-type and MEM alpha-type medium. Among these, there can also be mentioned for example the DME/F12 and DME/MCDB 202 mixtures. A basic nutrient medium which is particularly suitable during the culture of progenitor and/or stem cells, also comprises 4.5 g/l glucose and 3.7 g/l bicarbonate. Another example of preferred medium is the medium MCDB 120 modified by substituting the L-valine by the D-valine.

It is possible to use serum of animal origin (for example bovine or equine) in the composition of the present invention, but serum of human origin which can be obtained from PAA Laboratories is preferred for the purpose of avoiding any health risk of contamination in humans by serum of animal origin. Instead of the serum, it is also to use a serum fraction (constituted by one or more sub-elements of the serum) which is commonly obtained commercially (such as human albumin or transferrin). It is particularly advantageous of reduce the concentration of human serum so far as possible during the cell culture given that, unlike animal serum which is abundant and relatively easy to acquire, human serum is obtained from a more limited “source”, i.e. the patient and it is desirable to limit the number of blood collections and to take as little blood as possible. An embodiment according to the invention consists of using serum or serum fraction of human origin at a concentration of less than 5%, and more preferentially at a concentration comprised between 1 and 3%.

FIG. 1 surprisingly shows that the addition of the mixture M to human serum (C) makes it possible to obtain a production of myoblasts 3 times better than with HS (human serum) alone (A). Examples 2 and 3, in which the human and foetal calf serums respectively are supplemented, illustrate this in more detail.

The cell culture medium composition also contains insulin or insulinomimetics. Among the latter, hormones belonging to the class of the somatomedins or insulin-like growth factor, such as IGF 1 and IGF 2 or metals such as vanadate which inhibits a specific phosphatase group are found.

In the composition according to the invention, at least one antioxidant and/or one vitamin should be added to the culture medium. Among the antioxidants, N-acetyl-cysteine at a concentration comprised between 0 and 10 mM or selenium is preferred. Generally, selenium is used at a concentration comprised between 0 and 1 mM in the form of sodium selenite or selenomethionine (Sigma). It will be noted that the term “antioxidant” also refers to a culture condition in which oxygen-reduced partial pressure is used.

Among the vitamins, ascorbic acid at a concentration comprised between 0 and 1 mM or nicotinamide at a concentration comprised between 0 to 100 mM can be used. Vitamin E can also be used. Nevertheless, ascorbic acid is the preferred vitamin since it produces the best results as shown by Example 4.

Apart from serum combined with the compounds previously described, it is possible to add one or more compound(s) belonging to the class of FGF growth factors to the culture medium of the invention. These factors allow the cells in culture, in particular stem or progenitor cells to proliferate as well as to differentiate in specific fashion. This class of growth factors includes the bFGFs, FGF-2 to 10. Generally, these growth factors are used at a concentration between 0.1 ng/ml and 100 ng/ml.

According to an embodiment of the invention, at least one glucocorticoid can be added to the culture medium. These hormones act inter alia on the metabolism of the glucides. Natural or semisynthetic glucocorticoids can be used, i.e. hydrocortisone, dexamethasone, prednisolone or triamcinolone. Dexamethasone (Dex) is the preferred glucocorticoid. As shown by Example 5, the glucocorticoids have a stimulant and specific effect on cell growth.

Another embodiment of the invention consists of using one or more additional additives chosen from lipophosphatidic acid, the growth factors EGF, PDGF, heregulins, thrombin (IL6IL8, IL-15), LIF and thyroid hormones (including T3, T4).

It is also possible if appropriate to add transferrin as a protective factor against the heavy metals. Other hormones or active molecules can be included in the culture medium composition such as the hepatocyte growth factor, HGF/SF, and the different factors characterized such as LIF, VEGF, SCF, TGFb, TNFa, thrombopoietin or growth hormone.

Progestogens and derivatives (such as progesterone), oestrogens and derivatives (such as oestradiol), androgens and derivatives (such as testosterone), mineralocorticoids and derivatives (such as aldosterone), the hormones LH, LH-RH, FSH and TSH, retinoic acid and its derivatives, calcitonin, the prostaglandins E2 and F2/alpha or parathyroid hormone can also be used.

According to an embodiment of the invention, the composition defined above is quite particularly suitable as culture medium for progenitor and/or stem cells originating from muscle tissues.

According to the invention, it is preferable to use human serum which is autologous with the progenitor and/or stem cells cultured as this makes it possible to eliminate any risk of contamination which exists during the use of a heterologous serum. In this case the consequence is that treatment of the serum before its use within the framework of cell culture is no longer necessary.

The invention also relates to a process for producing myoblasts during which the muscle progenitor and/or stem cells are cultured on a culture medium the composition of which has been defined above. This production process can be divided into the following phases:
extraction: the cells are obtained from muscle tissues for example by enzyme treatment,
amplification: the cells obtained during the preceding step are cultured, they undergo a selective growth,
freezing of the cells originating from the amplification (if appropriate),
characterization of the cells originating from the amplification before their reimplantation in the patient.

Prior to this production process, a muscle biopsy is carried out in order to harvest the progenitor and/or stem cells. It takes place by incision under local anaesthesia. The size of the sample is approximately 1 g, from which it is possible to extract 10⁶ cells. Once the biopsy has been carried out, the tissue is placed in the protective medium. This protective medium essentially consists of the basic nutrient medium mentioned previously, to which antibiotics can be added, such as gentamycin, which is preferred to penicillin derivatives for its less allergic character; protective factors such as camitine (1 mM), insulin (10 μg/ml), dexamethasone (5.10⁻⁹ M), ascorbic acid, nicotinamide and trealose. The temperature must be below 25° C. and above 4° C. It is preferable for the volume of transport medium to be at least 10 times greater than the volume of the muscle tissue and for the transport time not to exceed 24 hours. The cells can in particular be obtained from the vastus extemus, vastus internus, biceps, quadriceps, tibial, gastrocnemii, peroneus, deltoids, large dorsal, stemocleidomastoid, intercostal, omohyoid, abdominals or from the psoas. A step of mincing can be carried out in order to allow better subsequent enzymatic dissociation. This consists of cutting the biopsy into sections of a size preferably smaller than 0.5 mm placed in a suitable culture medium. The mincing can be carried out manually using fine scissors. The slicing can also be carried out in assisted manner, using for example cutting mills powered by electric or mechanical energy. An example of such a useable mill is the Medimachine (distributed by Becton-Dickinson).

An embodiment of the invention consists of extracting cells from the muscle tissues. In fact, the muscle tissues are constituted by muscle fibres, within which the satellite cells are situated beneath the lamina basalis of the latter. The step of dissociation of the muscle fibres and separation of the satellite cells makes it possible to isolate the latter. The step of dissociation preferred according to the invention consists of the use of extracellular matrix digestion enzymes. The choice of the enzymes and their concentrations used for the dissociation of the muscle fibres and satellite cells of the tissues sampled is guided by the study of their enzymatic effectiveness, the criteria sought are the lowest possible concentration of enzyme and a minimum incubation time for similar effectiveness. The yield of cells obtained after filtration depends in part on the quality of the enzymatic dissociation step. Digestion enzymes which can be used in the process of the invention alone or in combination are, for example, all the collagenases, including the partially purified types IA, S and H, as well as the purified form marketed under the name of Liberase by Roche-Boehringer, pronase, or the trypsins, of all origins, in solution in buffers containing or not containing EDTA, dispases (also known as proteases), the elastases, or also the hyaluronidases. In particular the trypsin-collagenase or pronase-collagenase enzymatic combinations are suitable as the shown by the results of Example 1. Among the latter, the pronase-collagenase combination is preferred, as these are enzymes of non-extractive origin, thus making it possible to avoid any health risk of contamination by the prion or viruses. It is also possible to use collagenase as single enzyme. It is preferable to carry out this extraction step by a sequential process in order to minimize the time of exposure of the cells to the enzymes. It is also desirable for the duration of the enzyme treatments not to exceed 10 minutes and to use a treatment temperature comprised between 20 and 25° C. For all this step, the medium used is the protective medium. The inhibition of the action of the enzymes is carried out by dilution, washing and centrifugation.

Variants of the extraction treatment are applicable. On the one hand, the dissociation step can be carried out in two phases; a first incubation in the presence of collagenase and a second incubation in the presence of trypsin. On the other hand, it is possible to complete the enzymatic dissociation by a mechanical dissociation by aspiration and delivery of the suspension through a pipette.

It is then possible to monitor the effectiveness of the enzymatic digestion by microscopic observation of the cells released from the tissue fragment. By this observation, it is possible to note the presence of cells of various size, of red blood cells and of sarcomere fragments. These sarcomere fragments are good indicators of the effectiveness of the enzymatic digestion of the muscle tissue. It is recommended to again subject the tissue fragment not digested by the enzymes to a new enzymatic digestion according to the same treatment as described previously. It is particularly appropriate to repeat the operation 5 times. The cells can be frozen at this step (before culture) according to a protocol well known in the field of the art.

The invention also relates to the process for producing myoblasts during which the cell amplification step is carried out using the culture medium as already described. At the end of this amplification phase, a cell population of myoblasts is mostly obtained, i.e. in which a minimum of 70% myoblasts is found. This cell growth step is followed by a differentiation step: thus the growth medium previously described is replaced by a differentiation medium an example of which is provided hereafter (Example 6).

In order to improve the growth of the myogenic precursors, it is common in the field of cell culture to use collagen or its derivatives such as gelatin. These substances are obtained by extraction from bovine carcasses, which poses a health risk problem of contamination, for example, by the prion. The invention therefore proposes resolving this problem by using a protein which is obtained by genetic engineering. A commercially available molecule called Pronectin F, which is a polymer of the RGDS fragment of fibronectin, is quite particularly suitable. Having an effectiveness comparable to gelatin for the growth of human cells such as those which are precursors of muscle tissue, this protein can then be used within the framework of the invention as a substrate. It is also possible to use L-lysine or D-lysine polymers.

It is preferable for the cells to be cultured in a reactor suitable for the culture of adherent cells. In order to avoid the constraints of checking the stirring speed, its regularity and of the homogeneity of the preparations, the culture reactor is preferably static. It must have a large culture surface area compared with standard supports (Petri dishes, flasks) so as to harvest a large cell population in a few days. An example of such a culture reactor is the plate culture device (single, double and/or multi-stage).

The culture device which can be used in the process also makes it possible to carry out the sampling of the cells in sterile manner. This makes it possible to carry out samplings necessary for the identification of the cell types present in the different culture stages by analysis of specific markers. It allows the emptying of the media, the washing and the separation of the cells and finally their harvesting in sterile manner.

Bags and especially suitable sterile tubes can be used linking the bags to the reactor in order to allow the decantations of the media or the harvesting of the cells. This device thus makes it possible to carry out a large number of operations in a closed system. Depending on the desired cell population, the number of culture days varies from 0 to 45 days.

Moreover, the culture can be continued by standard expansion or perfusion techniques for a duration which can be up to several months.

In order to increase the number of cells harvested, one or more expansion phases are possible. The expansion phases include a step of separation of the cells, of washing of the cells and re-culturing on a larger culture surface area, the solutions and enzymes used for carrying out these steps being well known to a person skilled in the art.

In particular, the process of the invention comprises at least one cell expansion phase. Such a process makes it possible to multiply the number of cells whilst ensuring the differentiation of the initial progenitor and/or stem cells mostly to myoblasts at the end of each expansion culture. According to an embodiment of the invention, freezing of part of the cells having undergone the amplification step is carried out. A freezing protocol is provided in Example 8. For example, ⅕ of the culture can be frozen, i.e. approximately 2.10⁵ cells, the remaining ⅘ being subjected to a cell amplification process. In order to allow the use in time of the cells thus prepared, it can be advantageous to freeze them under conditions such that the subsequent thawing allows a sufficient survival of the cells, preferably more than 90%. By way of example, the cells are suspended in the freezing medium. These freezing medium compositions are typically DME/F12 medium with 1 mM L-carnitine, 0.2521 mM of ascorbic acid, 5.10⁻⁹ M dexamethasone, 10 μg/ml of insulin and 2% of human serum and transferred into two sterile freezing bags, at a concentration comprised between 10⁵ and 10⁷ cells/ml or in tubes of cryofreezing at a concentration comprised between 10⁵ and 10⁷ cells per ml. Under these conditions the preserving agent is DMSO at a concentration of 10%. Trehalose L-arginine (up to 0.5 M) can be added to this freezing medium. By immersion of the cells in this diholoside, this makes it possible to improve the preservation of the latter. The freezing is carried out using a device (Digicool or Nicool) ensuring a controlled progressive drop in temperature. The cells are stored in liquid nitrogen up to the time of thawing. A thawing of the cells frozen after culture can be carried out for example with a water bath at 37° C. The cell preparations are washed two times using an isotonic saline solution. The rinsings are carried out by sterile connection to the bags of isotonic solution and to the emptying bags. An aliquot is reserved for estimation of the cell viability and quality.

After cell amplification, a separation of the cells by enzymatic digestion should be carried out. During this step and for the purpose of reducing the health risks, it is advisable to use trypsin of recombinant origin which is commercially available.

Before carrying out cell transplantation within the framework of future clinical uses, it can be preferable according to the invention to characterize at molecular and functional level, the cell suspension obtained by the myoblast production process described above. This characterization can be carried out by analysis of cell markers by flow cytofluorimetry or FACS, after marking of the surface antigens or of any antigen specific to the different cell types to be analyzed. In the present text, the term “cell markers” indicates any cell antigen making it possible to supply information alone or in combination with other markers on a cell type.

This characterization can be undertaken at protein level using other cell markers such as:
P-Cam as an endothelial cell marker
N-Cam as a neuronal and muscle cell marker
“Smooth muscle actin” as smooth muscle marker
GFAP as glial cell marker
MyoD Myf5, Pax3, Pax7, C-met and M-cadherin, N-cam as muscle cell markers
Scal+, C-Kit, CD45, CD34 and CD56 as stem cells markers
PCNA, P21, P16, Ki67 as cell cycle markers.

This characterization can also be carried out at transcription level by the use of DNA chips (gene array) containing oligonucleotides coding for cell genes (for example, specific transcription factors and cell cycle machinery factors) making it possible to identify the cells in the cell suspension.

The obtaining of a cell population of a high degree of purity can prove necessary for certain uses as cell therapy product. It appears clear that a person skilled in the art can implement the different techniques proposed in the state of the art in order to selectively screen said cells. By way of example, there can be mentioned the techniques of screening by cloning, by flow cytofluorimetry or also by immunoaffinity or immunomagnetic columns using antibodies specific to the cells in question. To this end, both molecular characterizations and functional biological characterizations should be used. The cell markers chosen make it possible to identify the precursor cells of the muscle fibres. This identification is done not by a single marker but by a combination of markers. These are membrane markers such as N-Cam, Vla4, M-cadherin, integrins, CD56, cytoplasmic markers such as desmin and nuclear markers such as pax 7 and myoD. In order to increase the colonization and growth ability once implanted in the organism the cells used for cell therapy are positive for markers of the cell cycle machinery such as Ki67, PCNA and negative for cell cycle inhibitors such as P21 and P16. On the other hand these cells are negative for terminal markers of terminal muscle differentiation such as myogenin and troponin T (TNT). Analysis of cell functionality is carried out by biological tests in culture. The purpose of these tests is to determine in a cell sample the ability to form colonies in culture and the frequency of myogenic colonies. This test can be carried out, either after cell extraction, or before freezing, or after freezing. The principle is based on the analysis of low-density growth and the development of the cell phenotype by the use of specific differentiation medium. It should be noted that the cell seeding density must be kept as low as possible. Beforehand, the progenitor/stem cells are subjected to a growth phase, followed by a cell differentiation phase. The resulting cells are then fixed by an alcohol solution, stained with giemsa according to a protocol well known in the field of the art then photographed by a digital device. Within the framework of the functionality test, the latter are then subjected:
to macroscopic observation which makes it possible to count the total number of colonies and thus determine the percentage of cells which have a clonal growth potential,
to microscopic observation which makes it possible to determine the number and percentage of colonies of muscle precursors. The colonies of muscle precursors form, by cell fusion, polynucleate fusiform cells, the myotubes which will form muscle fibres in the organism.

This functional test is illustrated by Example 6. The present invention also covers any cell population which is contained in the culture medium as defined above. By cell population is meant any non-pure cell population, containing in general a dominant cell type and one or more minority cell types. This embodiment therefore relates to a population mostly of progenitor and/or stem cells (i.e. before the amplification phase has taken place), on a population enriched with myoblasts (following the cell amplification step), but also on an “intermediate” population, which corresponds to the case where no category of these cells is in the majority, i.e. during the amplification process. It can therefore be a mixture of different cell types.

The invention relates to the use of a cell population the dominant cell type of which is constituted by myoblast cells in the preparation of a cell therapy product for the reconstitution in humans of skeletal, cardiac and visceral muscle tissues and of vascular tissues.

According to a preferred embodiment, the population of myoblasts as a product of cell therapy is used for treating urinary incontinence in men or women. The latter can have as origin an insufficient pressure to close the urethra, the normal resistance of the urethra being half due to the smooth sphincter and half due to the striated sphincter of the middle urethra.

This cell therapy product can also be used to treat incontinence following prostate cancer treatment as well as innate or acquired muscular dystrophy. In dystrophy patients, the transplantation of myoblasts allows the restoration of dystrophin expression. It consists for example of injecting the cells of muscular origin obtained by a process of the invention directly into the skeletal muscle or into the general circulation using a needle.

A product of cell therapy suitable for human administration comprises an isotonic solution in which the cells are resuspended. It is preferable for this solution to be free of the toxic components present in the freezing media.

In the case of the treatment of urinary incontinence, this consists in particular of injecting, using a needle, a population of cells, the dominant type of which has the characteristics of myoblast cells, obtained and prepared as a cell therapy product, directly into the urethra or the rhabdosphincter, for the purpose of improving the function of the urethral closing mechanism. The number of cells injected is comprised between 10⁵ and 10⁷ cells.

It is also possible to control the injection of the myoblasts by a transurethral ultrasound probe, and to measure the urethral pressure before and after the injection, thus making it possible estimate the changes in urethral closing pressure using MRI in particular.

During the cell culture and expansion phases of the processes provided by the invention, a step of genetic modification of the cells by transfection of a heterologous nucleic acid can be carried out. The nucleic acid is chosen so as to allow the expression of a polypeptide or of a protein in the transfected cells. The transfected cells are then transplanted and allow the delivery of the polypeptide or of the protein expressed starting with the heterologous nucleic acid, said polypeptide or protein being a biologically active product. The invention thus relates to the use of a population of cells as a cell therapy product as a platform for delivery of a biologically active product. In order to genetically modify the precursor cells, it is preferable to use a viral approach which makes it possible to modify the cells in culture rapidly and effectively. The Moloney-type retroviruses are in this case particularly effective. It is possible to insert into this virus a molecular marker such as for example a GFP-type fluorescent protein (Example 7). The cells thus modified represent a tool for tracing the cell fate once introduced into the animal.

Another embodiment according to the invention consists of using the population of myoblasts in the toxicological and/or pharmacological screening. The objective is to shorten the development and preclinical and clinical test phases as much as possible in order to respond as rapidly as possible to the needs of patients. In fact, it is advantageous of use this population of cells as a “model” in the development of medicaments, thus making it possible to carry out high-throughput screening. It will then be possible to clarify the mechanisms at the origin of the diseases and to find the therapeutic targets or candidate molecules for becoming active ingredients. This screening can also serve in toxicology, in particular for studying medicament interactions.

The specialist in pharmacology/toxicology knows well how automated techniques are implemented, and will be able to select the molecules of interest, as a function of the target to be reached, from libraries of several thousands of new molecules or already utilized as medicaments for other pathologies. A preferred embodiment according to the invention consists of using pharmacological/toxicological screening in order to detect target molecules involved in rhabdomyolysis, i.e. lysis of striated muscles.

In fact fatal accidents with cholesterol synthesis inhibitors of the family of the statins (HMG-CoA reductase inhibitors) have placed muscle tissue at the forefront as a toxicological target. The poor evaluation of this risk known to Bayer for Cerivastatin has had considerable human and economic consequences.

A large number of drugs are capable of leading to myopathies. In acute and serious cases, considerable lysis of the muscle tissue (rhabdomyolysis) is experienced, the mechanism of which is still poorly understood. Several hypotheses have been put forward. Certain of these refer to an increase in the membrane permeability and others to anomalies at the level of the mitochondria.

In the great majority of cases, accidents occurring in the context of polychemotherapy suggest the role of interactions of medicaments (macrolides, immunosuppressors, anticancer agents, fibrates, cocaine, HIV antiproteases, anaesthetics etc.).

Among the numerous substances involved: the P450 cytochromes, molecules involved in apoptosis such as BCL2, antioxidants, proteins of the NFKb complex, PPARs or surgical interventions.

With the exception of acute accidents, threatening the vital prognosis (rhabdomyolysis), the clinical signs of muscle impairment are debility, muscle pain (myalgia), fatigability, cramps and the biological signs in addition to the acute accidents CPK levels are very frequently normal. In the case of the statins, recent data indicate that patients presenting with muscle impairment show neither correlations between the plasma level of this pharmacological agent and the toxic impairment, nor CPK elevation. In these cases, histology reveals modifications of the mitochondria (puff) and accumulations of lipid droplets in the muscle tissue.

Among the potential target molecules, there can be mentioned: HMG-coenzyme A reductase inhibitors, creatine kinase, the statins, fibrates, anaesthetics, heroin, the macrolides, cyclosporin as well as their derivatives.

The following section presents detailed examples intended to illustrate the present invention. However, the latter is not limitative in the sense a person skilled in the art can, with knowledge of the field, supply a few variants which are also covered by the invention.

### EXAMPLE 1

Cell Extraction from a Muscle Tissue Biopsy in the Absence of Proteins of Extractive Origin.

This experiment involves comparing the effectiveness of different cell-extraction protocols. The reference protocol used a mixture of two enzymes (trypsin and collagenase). The trypsin is of porcine origin and the collagenase is of bacterial origin. The action of these enzymes is stopped by the presence of foetal calf serum. In the following protocols, the enzymes of bacterial origin were chosen and the foetal calf serum was eliminated.

The progenitor/stem cells used originate from a biopsy of adult ewe muscle tissue.

The cell extraction protocol is of sequential type. The action of the enzymes is inhibited by dilution, washing and centrifugation. The duration of the enzyme treatments does not exceed 10 minutes. The treatment temperature is situated between 20 and 25° C. The muscle tissue (1 g of tissue after mincing) is brought into the presence of the enzyme solution (10 ml) i.e. at least 10 times greater than the volume of the muscle tissue. The enzyme solution is constituted by a combination of collagenase (0.5 mg/ml) and trypsin (1 mg/ml) without addition of serum, or a combination of collagenase (0.5 mg/ml) and pronase (1 mg/ml) with or without addition of foetal calf serum, these enzymes being solubilized in DME/F12 to which 15 mM Hepes is added.

The supplemented basic medium (without serum) used for the cell extraction is DME/F12 to which 15 mM Hepes is added, human insulin at 10 μg/ml, FGF-2 at 10 ng/ml, dexamethasone at 5.10⁻⁹ M, ascorbic acid at (0.252 mM) and L-carnitine at 1 mM. After 10 minutes of contact with the enzyme solution, the mixture (enzyme solution and tissue fragment) is diluted in a volume of 30 ml in order to inhibit the enzymes then subjected to slow centrifugation (less than 10 g per 3 minutes). By this process, the supernatant which contains the cells extracted by the first enzymatic digestion and the remaining tissue fragment are recovered. In order to recover the cells from the first digestion, centrifugation at 200 g is carried out for approximately 3 minutes. The cells thus obtained are resuspended in the medium without serum. The effectiveness of the enzymatic digestion is monitored by microscopic observation of the cells released from the tissue fragment. The remaining tissue fragment is again subjected to enzymatic digestion according to the same protocol. This operation is repeated five times in succession.

The substrate used for the cell attachment is bovine gelatin. DME/F12 supplemented by 20% foetal calf serum, 10 μg/ml insulin, 5 to 10⁻⁹ M dexamethasone and 10 ng/ml FGF-2 are used as culture medium. The culture conditions are the following: the temperature is 37° C. under a humid atmosphere, 20% oxygen and 5% carbon dioxide. The culture time is 7 days. The cells are fixed with an alcoholic solution and stained with giemsa stain. The dishes are then photographed.

The results indicate that the number of cells observed under the three different extraction conditions: trypsin+collagenase without bovine serum, pronase+collagenase with bovine serum (FCS) and pronase+collagenase without bovine serum (FCS) after one week of culture are similar, as is their differentiation potential. This example shows the effectiveness of the cell extraction techniques using enzymes of non-extractive origin such as pronase and/or collagenase.

### EXAMPLE 2

Effect of the “Supplementation” of Human Serum on the Amplification of Human Muscle Precursor Cells.

In this experiment the human cells originate from a biopsy from a normal subject.

The cell extraction step is carried out as previously.

In a first phase, the cells are amplified in culture in the presence of human serum (PAA Laboratories) then seeded under the different conditions described. The growth factors FGF2, EGF, PDGF A/B are produced by Preprotech and the thrombin is obtained from Sigma.

During the cell culture, the following are used:
2 multi-well plates with 12 wells (TPP)
bovine gelatin (Merck)
10,000 cells/well
1 ml of medium/well

The different culture media are prepared in the following manner: the basic nutrient medium is DME, to which the following are added:
no supplement (O)
1% human serum (1% HS)
5% human serum (5% HS)
Mixture M: FGF-2 (10 ng/ml)+insulin (10 μg/ml)+PDGF (1 ng/ml)+EGF (10 ng/ml)+dexamethasone (5.10⁻⁹M)+thrombin (I unit)
1% human serum+mixture M (1% HS+M)
5% human serum+mixture M (5% HS+M)
20% foetal calf serum (20% FCS)

It is to be noted that the mixture M contains no proteins of animal origin.

A second change of medium is carried out 3 days after the first. After 3 days of culture (total of 7 days), fixation and staining with giemsa are carried out. The number of cells fixed and stained is determined.

The results (expressed as number of cells per well) are indicated in Table 1 below:

TABLE 1
Effect of supplementing human serum (HS) on cell growth

| Culture medium tested | Number of cells/well |
|---|---|
| 0 | 10⁴ |
| 1% HS | 5 × 10⁴ |
| 5% HS | 7 × 10⁴ |
| M | 4 × 10⁴ |
| 1% HS + M | 23 × 10⁴ |
| 5% HS + M | 31 × 10⁴ |
| 20% FCS | 11 × 10⁴ |

According to these results, it is observed that the combination of the cocktail of growth factor and human serum makes it possible to obtain growth three times greater than that obtained in the presence of non-supplemented foetal calf serum. Under these conditions the amplification factor is greater than 30 after one week of growth. Surprisingly, it is important to note that human serum at concentrations of 1% and 5% supplemented by the mixture M vigorously stimulates cell proliferation since at these weak concentrations, a doubling and a tripling of the number of myoblasts compared with the non-supplemented 20% foetal calf serum are obtained, respectively. Finally, the serum supplemented by the mixture M improves the proliferation more than 4-fold in comparison with the non-supplemented serum.

### EXAMPLE 3

Improvement of the Growth Potential of Muscle Cell Precursors in the Presence De Supplemented Foetal Calf Serum.

In this experiment, the cell extraction and amplification steps are similar to those described previously. However, the culture parameters used are defined as follows: normal human cells are chosen, obtained at passage 7 after the cell extraction. The temperature is 37° C. in a humid atmosphere with 20% oxygen and 5% carbon dioxide. The cell density is 10³ cells per culture dish. The substrate used is gelatin.

As culture medium for the phase growth, the following are tested:
DME/F12 to which 20% foetal calf serum is added,
DME/F12 to which 20% foetal calf serum supplemented with insulin (10 μg/ml), ascorbic acid (0.252 mM), FGF-2 (10 ng/ml) are added,
PDGF (1 ng/ml), EGF (10 ng/ml), thrombin (1 unit), LPA (5 mM).

The duration of growth is 10 days and 7 days respectively with changes of medium every three days.

After fixation in alcohol, and staining with giemsa, a digital photograph of the petri dishes is taken.

The results are recorded in Table 2 below.

TABLE 2
Effect of supplementing foetal calf serum (FCS) on cell growth.

| Culture medium tested | Number of cells/well |
|---|---|
| 20% FCS | 11.10³ |
| 20% FCS + insulin + ascorbic acid + | 54.10³ |
| FGF + PDGF + EGF + thrombin + LPA | |

According to the results obtained, a very considerable improvement is surprisingly observed in the number of human muscle cells following the addition of the abovementioned cocktail compared with foetal calf serum alone, since this number of cells is increased by a factor of 5 over a growth period of only 7 days.

### EXAMPLE 4

Effect of Ascorbic Acid and Nicotinamide on the Amplification of Human Muscle Precursor Cells.

In this experiment the human cells originate from a biopsy from a normal subject aged 16 years.

The extraction protocol used is identical to that of Example 1. In the amplification step, the procedure as in the preceding example is followed, except that DME/F12 supplemented by either:

2% human serum+mixture M (insulin (10 μg/ml)+dexamethasone (5.10⁻⁹ M)+FGF-2 (10 ng/ml)+EGF (10 ng/ml)+thrombin (1 unit) (designated as 2% HS+M)

the preceding supplemented serum (2% HS+M) to which ascorbic acid at a concentration of 0.252 mM is added

the supplemented serum (2% HS+M) to which nicotinamide at a concentration of 10 mM is added

or the supplemented serum (2% HS+M) to which ascorbic acid and nicotinamide at the preceding concentrations are added is used as culture medium.

During the 8 days of culture, 3 changes of culture medium are carried out. At the end of this period, the cells are stained according to the same operating process as previously.

The results (expressed in number of cells per well) are indicated in Table 3 below:

TABLE 3
Effect of supplementing human serum (HS) by ascorbic
acid and/or nicotinamide on cell growth.

| Culture medium tested | Number of cells/well |
|---|---|
| 2% HS + “M” | 10⁴ |
| 2% HS + “M” + ascorbic acid | 32 × 10⁴ |
| 2% HS + “M” + nicotamide | 8 × 10⁴ |
| 2% HS + “M” + ascorbic acid + nicotamide | 18 × 10⁴ |

The addition of ascorbic acid, used in this experiment as an antioxidant, makes it possible to double the number of cells amplified after a period of 8 days of culture. By using nicotinamide as another antioxidant, no positive effect on growth is observed. The addition of these two antioxidants produces an intermediate result, it allows an increase in the number of cells amplified but not to the same level as with ascorbic acid alone.

### EXAMPLE 5

Specific Effect of Glucocorticoids on the Growth of Muscle Fibre Precursor Cells

Rat cells obtained at passage 23 after the cell extraction are used. The incubation temperature is 37° C. in a humid atmosphere with 20% oxygen and 5% carbon dioxide. The cell density is 3.10³ cells in multiples of 12. The substrate is gelatin.

As culture medium for the growth phase, DME/F12 is used to which 20% foetal calf serum is added, supplemented by insulin (10 μg/ml) and FGF (10 ng/ml).

The following are added to this medium:
either dexamethasone at increasing concentrations (from 10⁻⁶ M to 10⁻¹⁰ M)
or steroid hormones (oestradiol, testosterone, progesterone, DEHA, SDEAH, aldosterone) alone or in combination, such as dexamethasone with antiprogestogen RU486, at a fixed concentration (10⁻⁷ M).

The duration of culture is 5 days without change of medium.

After alcoholic fixation and staining with giemsa, a digital photograph is taken.

The results are shown in FIGS. 2A and 2B.

According to these results, it is shown that the addition of dexamethasone very clearly improves cell proliferation and that its optimum concentration is comprised between 10⁻⁶ M and 5.10⁻⁹M. Moreover, the effect of this glucocorticoid is specific to the growth of muscle precursor cells, unlike the other steroid hormones tested which do not improve growth. Finally, the presence of an antiprogestogen such as RU486 eliminates the effect of the glucocorticoids.

### EXAMPLE 6

Functional Testing of Human Muscle Cell Precursors

Healthy human cells at passage 1 after extraction are used. The cell culture conditions are the following: the temperature is 37° C., humid atmosphere, 20% oxygen and 5% carbon dioxide. The cell density is 10³ cells per culture dish which originate from a muscle tissue of 100 mm. The substrate is gelatin. The culture medium used in this experiment for the growth phase is DME/F12 to which 20% foetal calf serum, 10 μg/ml human insulin, 5.10⁻⁹ M dexamethasone and 2.10 ng/ml FGF are added. The growth time is 9 days with changes of medium every three days. Then, DME/F 12 to which 2% human serum, 10 μg/ml insulin, 10 ng/ml EGF, and 5.10⁻⁹M thyroid hormone T3 are added, is used as differentiation medium. The differentiation time is 4 days with a change every 2 days. Then alcoholic fixation, and staining with giemsa are carried out, followed by taking a digital photograph of the dishes.

According to this experiment, macroscopic observation makes it possible to count 107 colonies. Among the latter, two types of colonies can be noted. The colonies of the first type are stained intensely and microscopic observation reveals the presence of numerous differentiated muscle cells, myotubes: these are colonies formed by muscle precursors. The colonies of the second type, paler under macroscopic observation, contain no myotubes: these are non-muscle cell colonies.

The results are the following: among 107 colonies in total, 91 colonies of muscle tissue precursor cells and 16 colonies of non-muscle cells are counted. Thus, overall, among 1000 cells seeded, 10.7% of the cells are capable of forming colonies and among the latter 85.6% are capable of forming colonies of muscle tissue precursor cells.

### EXAMPLE 7

Genetic Modification of the Muscle Fibre Precursor Cells.

In order to genetically modify the precursor cells, we used a Moloney-type retrovirus (MMLV) into which the sequence coding for the “green fluorescent protein” (GFP) has been inserted. Screening-infection is carried out by a packaging plasmid containing the sequences “gag” and “pol”, a plasmid containing the VSVg envelope, and a plasmid containing the GFP construction according to a protocol well known to a person skilled in the art.

Rat cells obtained at passage 21 after the cell extraction are used. The incubation temperature is 37° C. under a humid atmosphere with 20% oxygen and 5% carbon dioxide. The cell density is 2.10⁴ cells per 35-mm dish. The substrate used is gelatin.

As culture medium for the growth phase, the basic nutrient medium DME/F12, to which 20% foetal calf serum supplemented by insulin (10 μg/ml), dexamethasone (5.10⁻⁹M) and FGF (10 ng/ml) are added, is used.

The infection protocol is the following: the day following seeding of the cells, infection of the cells with the virus rMLV (VsVg)LTR-eGFP at a dose of 8.3.10⁶ ip/mL is carried out. The infection is carried out using 10 infectious particles per cell (M01).

The sample is diluted in a final volume of 5 mL for a 10-mm dish of the medium comprising:
DMEM/F12
polybrene (8 μg/ml) (molecule aiding the introduction of DNA into the cell)
insulin (10 μg/ml)
FGF (10 ng/ml).

The cells are incubated for 6 hours at 37° C. then the medium is replaced by 10 mL of DME/F12 supplemented by 20% FCS and insulin (10 ng/ml), dexamethasone (5.10⁻⁹ M) and FGF-2 (10 ng/ml).

On the 3rd and 7th days, the living cells are observed by microscopic photography with a fluorescence microscope. The myoblasts are counted, as well as the myotubules, which appear green and which have therefore been transfected by the virus.

As expected, the non-infected cells develop no fluorescence and a very large majority of the cells express GFP and thus appear green, under these conditions more than 90% of the cells express GFP. GFP is also correctly expressed in the myotubes which result from the fusion of the myoblasts. Under these conditions, the myoblasts, which are replicative cells, and the myotubes, which are differentiated cells, can be genetically modified and this modification is stable. The number of cells expressing GFP is not modified by culture passages. After reintroduction into the animal, the cells thus modified express GFP and can thus be observed. This tool is important for analyzing the fate and functions of the cells once reintroduced into the animal.

### EXAMPLE 8

Improvement of Cell Freezing Techniques by Use of Human Serum in Weak Concentration.

The cells used originate from a normal individual aged 16 years. They are cultured and harvested at passage 7.

During the culture phase, 2% human serum (HS) supplemented by 10 μg/ml insulin, 0.252 mM ascorbic acid, growth factors FGF (10 ng/ml), PDGF (1 ng/ml), EGF (1 ng/ml), as well as thrombin (1 unit) and LPA (5 mM) is used as medium.

The enzyme treatment is carried out as in Example 1, using trypsin-EDTA (PAA Laboratories) as enzyme. The treatment time is 10 minutes.

Once detached from their substrates, the cells are placed in the different freezing media which are the following, at a concentration of 10⁵ cells per ml:

DME/F12 medium alone or supplemented by:
90% FCS
10% FCS
10% HS
2% HS
5.10⁻⁹M dexamethasone
10 μg/ml insulin
0.252 mM ascorbic acid
dexamethasone+insulin+ascorbic acid (at the preceding concentrations)
2% HS+5.10⁻⁹ M dexamethasone
2% HS+10 μg/ml insulin
2% HS+0.252 mM ascorbic acid
2% HS+dexamethasone (5.10⁻⁹M)+insulin (10 μg/ml)+ascorbic acid (0.252 mM)

is added to a solution of 10% DMSO as cryopreserving agent and 90% foetal calf serum (FCS).

After 10 minutes at ambient temperature, the cells are gradually cooled down to a temperature of −80° C.

Thawing is carried out in an incubator or in a water bath at 37° C. The vial preserved in liquid nitrogen is placed in a culture incubator. After 5 minutes, the thawed cells are placed in a 10-ml centrifugation tube in the presence of: DME/F12 supplemented by pyruvate, antibiotics such as gentamycin and protective factors such as 1 mM L-carnitine, 10 μg/ml insulin, 5.10⁻⁹M Dexamethasone, 0.252 mM ascorbic acid. Centrifugation is carried out at 200 g for 10 minutes at ambient temperature. The cells thus thawed are cultured in multiples of 12 with gelatin as substrate, and 2% human serum (HS), HS supplemented by insulin (10 μg/ml), ascorbic acid (0.252 mM), and growth factors FGF2 (10 ng/ml), PDGF (1 ng/ml), and EGF (1 ng/ml), as well as thrombin (1 unit) and LPA (5 mM) as culture medium.

After 2 days of culture, the medium is changed using new multi-well dishes. At this stage, staining of part of the cells is carried out in multi-well dishes. The other part is subjected to a new culture phase for another 4 days and to a new change of medium. Staining is carried out 2 days later.

The results are recorded in Table 4 below:

TABLE 4
Effect of the type of freezing medium on the cell
growth (expressed as number of cells/well).

| Type of freezing medium | Number of cells/well |
|---|---|
| DME/F12 alone | 0 |
| DME/F12 supplemented by 90% FCS | 10,700 |
| DME/F12 supplemented by 10% FCS | 20,000 |
| DME/F12 supplemented by 10% HS | 18.750 |
| DME/F12 supplemented by 2% HS | 8,000 |
| DME/F12 supplemented by dexamethasone | 0 |
| (5.10⁻⁹ M) | |
| DME/F12 supplemented by insulin (10 μg/ml) | 0 |
| DME/F12 supplemented by ascorbic acid | 0 |
| (0.252 mM) | |
| DME/F12 supplemented by dexamethasone | 0 |
| (5.10⁻⁹ M) + insulin(10 μg/ml) + | |
| ascorbic acid (0.252 mM) | |
| DME/F12 supplemented by 2% HS + | 13,500 |
| dexamethasone (5.10⁻⁹ M) | |
| DME/F12 supplemented by 2% HS + insulin | 15,000 |
| (10 μg/ml) | |
| DME/F12 supplemented by 2% HS + ascorbic | 12,000 |
| acid (0.252 mM) | |
| DME/F12 supplemented by 2% HS + | 13,000 |
| dexamethasone (5.10⁻⁹ M) + insulin | |
| (10 μg/ml) + ascorbic acid (0.252 mM) | |

These results confirm that the freezing medium must contain serum or fractions of the latter such as albumin for good cell preservation. They also surprisingly show that the presence of the different additives such as insulin, dexamethasone and ascorbic acid make it possible to increase the effectiveness of freezing in the presence of a weak concentration of serum, and in particular of human origin. By these means, it is shown that it is possible, with a view to preserving good subsequent cell growth, to optimize the freezing medium by reducing the serum concentration whilst avoiding the risks of contamination by prions and viruses of animal origin.

### EXAMPLE 9

Selection and Amplification of Muscle Progenitor Cells from Biopsies.

It is possible to select and amplify the muscle progenitor cells present in the biological samples by culture techniques. From the cellular point of view, a muscle tissue biopsy is very heterogeneous. Both for cell therapy and for pharmacological and toxicological utilization this heterogeneity is a handicap.

The technique used is based on the construction of culture techniques which dissociate the muscle progenitor cell selection period from the muscle progenitor cell amplification period. A progenitor cell selection medium and subsequently an amplification medium are used.

The medium for positive selection of the muscle progenitor cells combines both agents which inhibit the growth of non-muscle cells and agents which stimulate the growth of the muscle progenitor cells. The first belong to the family of the glucocorticoids and the second are antioxidants and metals. In this selection phase, the cells originating from the muscle biopsy after enzymatic digestion are cultured at clonal density in the presence of the inhibiting agents and stimulating agents.

The amplification medium contains growth factors which make it possible to facilitate the growth of the cells selected. These factors belong to the family of the FGFs. In this phase, the cells can be cultured either at a low density or at a high density.

The protocol described in two steps makes it possible to obtain muscle cell populations enriched to more than 95%.

As for Example 6 the cells are seeded at clonal density. In this type of test, each cell gives rise to a cell colony the phenotype of which is analyzed.

The cells originate from a normal individual without muscle pathology. The cells are seeded at clonal density of 250 cells per 100-mm dish in 10 ml of culture medium. The following media are used for the selection period, Day 0:
DMEM/F12+FCS.
DMEM/F12+FCS+FGF.
DMEM/F12+FCS+Insulin+Dexamethasone+Selenomethionine+Ascorbic Acid.
DMEM/F12+FCS+FGF+Insulin+Dexamethasone+Selenomethionine+Ascorbic acid.

On day 3 for the four series the media are changed for the following identical medium: DMEMIF12+FCS+FGF+Insulin+Dexamethasone.

The medium is changed on day 6 and day 10 in the four series. On day 14 the medium is changed for a medium allowing the differentiation of the muscle cells composed of:
DMEM/F12+1% FCS+Fetuin+Insulin+EGF+T3.
On day 19 the cells are fixed and stained as described in Example 6.

The results obtained are the following:
The cells cultured in FCS provide 70 colonies/dish including 10% myogenic colonies.
The cells cultured in FCS+FGF provide 70 colonies/dish including 0% myogenic colonies.
The cells cultured in FCS+Insulin+Dexamethasone+Selenomethionine+Ascorbic acid provide 150 colonies/dish including 100% myogenic colonies.
The cells cultured in FCS+FGF+Insulin+Dexamethasone+Selenomethionine+Ascorbic acid provide 80 colonies/dish including 50% myogenic colonies.

The presence of the combination Insulin, Dexamethasone, Selenomethionine and ascorbic acid allows highly effective selection of the muscle progenitor cells.

### EXAMPLE 10

Cell Tests Predictive of Muscle Toxicity

In order to construct cell cultures allowing in vitro toxicological tests we used rat muscle and adipocyte cells in order to analyze the specificity of the muscle toxicity.

The conditions of the experiment are the following:

The origin of the cells and their type are: Rat (muscle cells) and Rat 160 mg (adipocytes). Their passage numbers are P9 and P4. The culture conditions are FCS+FGF+Insulin+Dexamethasone.

The enzyme treatment is carried out with Trypsin-EDTA (PAA), the treatment time being 5 minutes.

Centrifugation is carried out.

The handling conditions are the following: type of dish: 4 multiwell plates with 12 wells (TPP); substrate: Gelatin, density: 5,000 cells/well, the culture medium is DME/F12+20% FCS+FGF+Insulin+Dexamethasone+Statins (at concentrations of 0; 0.1; 0.5 or 1 μM).

The concentrations are FGF: 10 ng/ml; Insulin: 10 μg/ml, dexamethasone: 5.10⁻⁹ M.

The cells are thus cultured for 2 days then fixed, stained and analyzed.

This analysis reveals a preferential toxicity of Lovastatin for the muscle cells. At 0.5 μM, the muscle cells are very inhibited in their growth whereas the adipocyte cells are insensitive to it. FIG. 3 shows the results of the digital processing of the toxicity test results.

These experiments were reproduced with human muscle cells in order to test the toxicity of the commercial statins.

The experimentation conditions are the following:
the type of dish is 2 multiwell plates with 96 wells (TPP)
the cell density is 2,500 cells/well
the culture medium contains DME/F12+20% FCS+FGF+Insulin+

Dexamethasone+X.

X being chosen from:

Lovastatin at a concentration of 0; 0.01; 0.05; 0.1; 0.5 μM; or

Cerivastatin at a concentration of 0; 0.01; 0.05; 0.1; 0.5; 1 μM; or

Atorvastatin at a concentration of 0; 0.01; 0.05; 0.1; 0.5; 1 μM; or

Pravastatin at a concentration of 0; 0.01; 0.05; 0.1; 0.5; 1 μM; or

Fluvastatin at a concentration of 0; 0.01; 0.05; 0.1; 0.5; 1 μM; or

Simvastatin at a concentration of 0; 0.01; 0.05; 0.1; 0.5; 1 μM.

The experimentation procedure is as follows.

On day 1 the media are changed. On day 3 staining is carried out.

The total culture time is 5 days.

After aspiration of the culture medium, the cells are washed with PBS then fixed with 100% ethanol. 10 minutes later the cells are washed with water then stained with a solution of 10% Giemsa for 10 minutes. The final step is washing with water.

Images of the cells are obtained with an inverted microscope (Nikon) equipped with a digital camera and a motorized stage.

The digital processing is presented in FIG. 4. This figure reveals the high toxicity of Cerivastatin. The latter molecule proved to be the most toxic statin in human clinical use. This test therefore makes it possible to reveal the preferential toxicity of Cerivastatin for human muscle cells.

1. Cell culture medium composition containing:
(i) serum and/or serum fraction of human origin and/or of animal origin
(ii) insulin or a derivative of the latter
(iii) one or more compound(s) chosen from the class of antioxidants and/or vitamins.

2. Composition according to claim 1, in which human serum is used.

3. Composition according to claim 1, in which bovine serum is used.

4. Composition according to claim 1, comprising moreover one or more compound(s) chosen from the class of FGF-type growth factors.

5. Composition according to the preceding claim, in which the class of FGF-type growth factors is composed of bFGF, FGF-2 to FGF-10.

6. Composition according to one of the preceding claims, in which the insulin derivative is chosen from the class of the IGFs, and vanadate-type insulomimetics.

7. Composition according to any one of claims 1-2 and 4-6, in which the human serum concentration is less than 5% by volume, preferably between 1% and 3%.

8. Composition according to one of the preceding claims, which moreover comprises a glucocorticoid.

9. Composition according to any one of the preceding claims, said vitamin being ascorbic acid.

10. Composition according to any one of the preceding claims, said antioxidant being N-acetyl-cysteine and/or selenium.

11. Composition according to any one of the preceding claims, which moreover comprises lipophosphatidic acid and/or one or more compound(s) of the classes of the EGFs, heregulins, thrombin, PDGF, thyroid hormones and LIF.

12. Process for the culture of progenitor and/or stem cells, in which the composition according to one of the preceding claims is used as culture medium during the cell amplification step.

13. Process according to the preceding claim, in which a cell differentiation step is carried out before, during or after said cell amplification step.

14. Process according to claim 12 or 13, in which the human serum used is autologous with the progenitor/stem cells.

15. Process for producing myoblasts by implementation of the process according to one of claims 12 to 14.

16. Process for producing myoblasts according to the preceding claim, in which the progenitor and/or stem cells are obtained by a step of cell extraction from muscle tissues.

17. Process for producing myoblasts according to the preceding claim, said extraction step being carried out by enzymatic digestion.

18. Process for producing myoblasts according to one of claims 15 to 17, in which a harvesting and a separation of the cells obtained is carried out.

19. Process for producing myoblasts according to the preceding claim, in which said step of harvesting and separation of the cells is carried out by enzymatic digestion followed by centrifugation and/or filtration.

20. Process for producing myoblasts according to one of the claims 15 to 19, in which a functionality test is carried out on the suitability of the myoblasts for forming colonies.

21. Process for producing myoblasts according to one of claims 15 to 20 in which a characterization step is moreover carried out.

22. Process for producing myoblasts according to the preceding claim, in which cell cycle markers are used.

23. Process for producing myoblasts according to one of claims 15 to 22, in which a step of freezing of the myoblasts is carried out.

24. Cell population containing progenitor and/or stem cells and/or myoblasts in the culture medium according to one of claims 1 to 11.

25. Use of the myoblasts according to one of claims 15 to 23, said product being intended for cell therapy.

26. Use of the myoblasts according to the preceding claim for the preparation of a product intended for the functional treatment of the small muscles.

27. Use of the myoblasts according to claim 25 for the preparation of a product intended for the treatment of urinary incontinence.

28. Use of the myoblasts according to one of claims 15 to 23, said product being intended for gene therapy.

29. Use of the myoblasts by the process obtained according to one of claims 15 to 23 in toxicological and/or pharmacological screening.

30. Use of the myoblasts according to the preceding claim for detecting one or more substance(s) involved in rhabdomyolysis.
